(19) United States
(12) Patent Application Publication
(10) Pub. No.: US 2005/0196375 A1
(43) Pub. Date: Sep. 8, 2005

(54) Methods of applying antibiotic compounds to polyurethane biomaterials using textile dyeing technology

(76) Inventors: Matthew D. Phaneuf, Ashland, MA (US); William C. Quist, Brookline, MA (US); Michael Szycher, Lynnfield, MA (US); Martin J. Bide, Wakefield, RI (US); Frank W. LoGerfo, Cambridge, MA (US)

Correspondence Address: CLARK & ELBING LLP, 101 FEDERAL STREET, BOSTON, MA, 02110, US

(21) Appl. No.: 11/110,198

(22) Filed: Apr. 20, 2005

Related U.S. Application Data
(63) Continuation of application No. 09/834,978, filed on Apr. 13, 2001, now abandoned.
(60) Provisional application No. 60/197,278, filed on Apr. 14, 2000.

(51) Int. Cl. A61K 31/765; A61K 31/496; A61K 31/4709

(52) U.S. Cl. 424/78.3; 514/312; 514/253.08

(57) ABSTRACT

The invention provides urethane polymers bonded to therapeutically active compounds, such as antibiotics. The invention also features methods of applying therapeutically active compounds to polyurethane polymers using textile dyeing. These polymers may be used in a variety of clinical applications.

### CROSS REFERENCE TO RELATED APPLICATIONS

This application is a continuation of, and claims priority from, U.S. Ser. No. 09/834,978, filed Apr. 13, 2001, which claims the benefit of the filing date of U.S. Provisional Application No. 60/197,278, filed Apr. 14, 2000, each of which is hereby incorporated by reference.

### BACKGROUND OF THE INVENTION

Any invasion of the skin carries with it the risk of infection. This applies to simple surface wounds, some 4-6% of which become infected. Surgical procedures use a similar range of biomaterials for wound closure and dressings, and may also involve implantable devices (catheters, vascular grafts, heart valves). Infection of these materials is of major concern despite recent advances in sterile procedures used in the clinical setting. For example, around 50-100,000 indwelling vascular catheters become infected each year in the US with concomitant human suffering and cost implications The delivery of antibiotics to wounds in general has been the subject of study, and within the larger field of slow-release drug delivery systems, implantable biodegradable materials have been used.

Inoculation of the biomaterial presumably occurs at the time of implantation or as a result of transient bacteremia in the immediate post-operative period. Perioperative parental antibiotics, while having a defined role in wound infection prophylaxis often fail to permeate the avascular spaces immediately around prosthetic grafts and the carbohydrate-rich bacterial biofilm once pathogens have adhered. Staphylococcus aureus (S. aureus) is responsible for 65-100% of acute infection. These infections are typically quick to develop and generate an intense response by the body's defense mechanisms. An ever increasing problem, which has been documented both in animal models and in humans is the susceptibility of vascular prostheses to late infection. Staphylococcus epidermidis (S. epidermidis) recently emerged as the leading isolate from infected vascular conduits (20-60%) with infection appearing late after implantation. These cases are clearly not affected by low-level antibiotic transiently present at the time of operation, which may in fact lead to the development of resistant organisms.

Numerous strategies have been attempted in order to create an infection-resistant prosthetic graft surface. The simplest and most widely used approach includes dipping the biomaterials in an antibiotic solution immediately prior to implantation. It has been suggested that prosthetic, knitted Dacron grafts could be simply coated using antibiotics (such as, nafcillin, cefazolin, cefamandole), or a suspension of silver-pefloxacin and a silver-nalidixic acid analogue at the time of implantation to obtain an infection-resistant prosthesis.

Chelating agents have also been evaluated as a release system for antibiotics from a biomaterial surface. One approach which has been the subject of numerous investigations was the ionic binding of antibiotics by surfactants. Cationic surfactants such as tridodecylmethyl ammonium chloride (TDMAC) and benzalkonium chloride were sorbed at the anionic surface potential of a polymeric material, thereby permitting weak adhesion of anionic antibiotics to the surface. The selected antibiotic was then released upon contact with blood. Later, Greco found that the custom synthesized surfactant trioctadecylmethylammonium chloride (TOMAC) was superior to TDMAC in binding to the graft surface, resulting in antibiotic binding that was twice as effective with TOMAC. Silver was also examined as a release system for various antibiotics from graft surfaces, applied either as a chelating agent or alone due to its antimicrobial properties.

Binder agents have also been employed in order to create localized concentrations of antibiotic on the graft surface. These agents, which were either protein or synthetic-based, were embedded within the biomaterial matrix thereby either “trapping” or ionically binding the antibiotic. For example, an infection-resistant arterial prosthesis has been developed using a collagen-release system to bond amikacin on uncrimped filamentous velour prostheses. In addition, the efficacy of binding an antibiotic cefoxitin to a PTFE vascular prothesis via a glucosaminoglycan-karatin luminal coating has been studied. The basement membrane protein collagen has served as a release system for rifampin, demonstrating antimicrobial efficacy in a bacteremic challenge dog model, as well as, in early European clinical trials. Fibrin, either as a pre-formed glue or in pre-clotted blood, has been utilized as a binding agent for various antibiotics including gentamycin, rifampin and tobramycin. Levofloxacin has been incorporated in an albumin matrix and gelatin has been used as the release system for the antibiotics rifampin and vancomycin, with animal studies also showing efficacy in acute bacteremic challenges.

Synthetic binders have also been evaluated for antibiotic release as a replacement for the protein binders. Some synthetic binders were incorporated directly into the biomaterial matrix, in a similar fashion as the protein binders, permitting sustained release of a selected antibiotic over time. For example, a PTFE vascular graft was treated with a suspension of N-butyl-2-cyanoacrylate and tobramycin powder (antibiotic glue, ANGL). This study showed that ANGL could be effective in the prevention and treatment of prosthetic graft infection. A low infection rate was reported in clinical studies using a low concentration rifampicin soaking of partly cross-linked gelatin grafts. Another study on rifampicin-soaked, fully cross-linked, gelatin dacron reported equally good, six-month follow-ups on staphylococcal infections. Recent techniques have also utilized these types of binder materials as a scaffolding to covalently bind antibiotics to the biomaterial surface. Release of the antimicrobial agent was controlled by bacterial adhesion to the surface that resulted in antibiotic cleavage. This method promotes “bacterial suicide” while maintaining antibiotic, which is not needed to prevent infection, localized on the surface. Other techniques have involved incorporating the antibiotic either into the synthesis process of the polymer (Golomb et al., J. Biomed. Mater. Res. 25:937, 1991) or by embedding the antibiotic directly into the interstices of the material (Okahara et al., Eur. J. Vasc. Endovasc. Surg. 9:408, 1995).

There are several drawbacks to the technology currently available. For the chelation agents, 50% of the antibiotic has been shown to elute from the graft surface within 48 hours, with less than 5% remaining after three weeks (Greco et al. Arch. Surg. 120:71-75, 1985). While this antibiotic coverage is adequate for small-localized contaminations, large inoculums are not addressed. For the binding agents, antibiotic release may be quite varied depending on the rate of binder degradation or binder release from a surface that is under high shear stress from blood flow. Comparably, both types of surface modifications rely on exogenous material that may effect the overall healing of the graft surface, either by releasing toxic moieties or by promoting thrombogenesis. Thus, these potential complications have accentuated the need to investigate the basic interactions between antibiotics and fibers in order to create an infection resistant graft surface that is void of exogenous materials.

Noticeably all of the above work avoids the examination of any direct material/antibiotic interaction. In contrast to proteins which can remain active when covalently bound, bacteriocidal antibiotics must be released to regain their activity. Covalently bound bacteriostatic antibiotics may, however, retain the ability to inhibit mucin production, thus, preventing the growth of bacteria. Recent work has attempted to use direct interactions using dye-fiber interactions as a model in order to provide infection resistance without exogenous binders. The process of dyeing refers to an uptake of a compound that is dramatically in excess of the amount taken up by simple imbibition (absorption) of the solution containing the compound, and which extends throughout the solid substrate, not just on the substrate surface. In dyeing, a textile substrate is immersed in a bath containing dye. During the incubation, the dye will equilibrate between the bath ant the textile substrate, considerably in favor of the latter.

Dyes are colored organic materials that are soluble during application, have substantivity/affinity for fibers, and have “fastness” (resistance to removal or destruction) in subsequent use. Typical dyes have molecular weights of 300-900, and functional groups that confer both solubility and substantivity towards fibers. Many thousands have been commercialized. Dyes will “exhaust” from a bath preferentially into a fiber. The literature on dye-fiber interactions is extensive and the theoretical basis for dyeing is well established. The subject is discussed in more detail below. Parameters such as the diffusion coefficient of dyes in fibers, and the chemical affinity of dye for fiber can be measured (Nunn, The Dyeing of Synthetic Polymers and Acetate Fibers, Dyers Publication Trust, Bradford, UK, 1979; Johnson, Theory of Coloration and Textiles, Society of Dyers and Colorists, Bradford, UK, 1989; Shore, Colorants and Auxiliaries, Society of Dyers and Colorists, Bradford, UK, 1990).

Initial efforts in this regard examined the use of commercially available dyes as anchors for antibiotic molecules, and even the determination of antibiotic activity of some dyes. This approach was unrewarding. In contrast, the direct use of antibiotics was examined. Fluoroquinolone antibiotics are particularly suitable in such applications. They are stable to dry heat and to hot aqueous media; they also have structural features (solubility, molecular mass, and functional groups) that coincide with those of dyes (FIG. 1). The fluoroquinolones represent a relatively new class of antibiotics with outstanding therapeutic potential, attributable to their broad spectrum of antimicrobial activity and favorable tissue distribution. They are effective at low concentrations against most Gram-negative pathogens, as well as, Gram-negative and Gram-positive bacteria and are the drug of choice for many applications. Fluoroquinolones now extend to at least ten members, including ciprofloxacin, ofloxacin, norfloxacin, sparfloxacin, tomafloxacin, enofloxacin, lomefloxacin, pefloxacin, fleroxacin and DU6859a. The most common commercially available quinolones are ciprofloxacin (cipro) and ofloxacin (oflox). Phaneuf, LoGerfo, Quist, Bide et al. (Bide et al., Textile Chemist and Colorist 25:15-19, 1993; Phaneuf et al., J. of Biomedical Materials Res. 27:233-237, 1993; Ozaki et al., J. of Surgical Res. 55:543-547, 1993) applied cipro to Dacron graft via thermofixation, an application method founded on estabilished textile procedures. The graft was dipped in 5 g/l ciprofloxacin solution, squeezed to a wet pickup level of 65%, air dried and then heated at 210° C. for two minutes. This thermofixation method was compared with the dipping method and showed superior, sustained antistaphylococcal activity.

Because none of the current techniques has resulted in a satisfactory infection-resistant biomaterial, a new biomaterial is needed that has good tissue and blood compatability and causes a lower rate of bacterial infection.

### SUMMARY OF THE INVENTION

This invention features a method of applying a therapeutically active organic compound to a urethane polymer containing a functional group within the polymer backbone. This method includes incubating the polymer with the compound in solution (aqueous or organic) under conditions that result in reversible adsorption of the compound from solution by the polymer. In one preferred embodiment of the invention, the adsorption of the compound by the polymer involves dyeing.

By “dyeing” is meant an uptake of a compound that is substantially (more than ten times) in excess of the amount taken up by simple imbibition (absorption) of the solution containing the compound, and which extends throughout the solid substrate, not just on the substrate surface.

In other embodiments, the compound is directly bonded to the polymer. By “directly bonded” is meant chemically bonded to the polymer without an intervening chemical moiety. For example, the compound may non-covalently bond to the polymer through an interaction such as an ion-ion force, dipole-dipole force, hydrogen-bond, van der Waals force, electrostatic interaction, or any combination of these interactions.

Preferably, the polymer and the compound are incubated at a temperature between 35 and 90° C. for at least 1 hour. For a urethane polymer containing carboxylic acid functional groups, the pH of the solution containing the polymer and the compound is greater than 7.5. For a urethane polymer containing amino functional groups, the pH of the solution containing the polymer and the compound is less than 7.5. Other preferred functional groups contained within the polymer backbone are sulfo or hydroxyl groups. In various embodiments, at least 1, 5, 10, 15, 20, 30, 40, 50, 60, 70, 80, 90, 95, or 100% of the carboxylic acid, amino, sulfo, or hydroxyl groups in the polymer backbone are bonded to the compound. In one preferred embodiment of the invention, the concentration of the compound is at least 0.5% weighed polymer/fiber and the solution has a liquor ratio of 10 or greater. In other embodiments, the compound remains bonded to the polymer for at least one day in phosphate-buffered saline at pH 7.4 and 37° C. or in vivo. In yet other embodiments, the compound remains bonded to the polymer for less than ten days in phosphate-buffered saline at pH 7.4 and 37° C. or in vivo. In still other embodiments, the compound remains bonded to the polymer for a period between one and ten days, inclusive, in phosphate-buffered saline at pH 7.4 and 37° C. or in vivo. If desired, the period of time during which the compound is released from the polymer may be increased by increasing the percentage of functional groups in the polymer or by increasing the thickness of the polymer. The percent by weight of functional groups in the polymer (calculated by determining the weight of the functional groups divided by the total weight of the polymer) may be increased by increasing the ratio of the number of molecules of chain extender to the number of molecules of diol and diisocyanate used to synthesize the polymer. In particular embodiments, the percent by weight of the functional groups in the polymer is between 1 and 30%, inclusive. In other embodiments, the percent is contained in one of the following ranges: 1 to 5%, 5 to 10%, 10 to 15%, 15 to 20%, 20 to 25%, or 25 to 30%, inclusive. In other embodiments, the percent of carboxylic acid groups in the polymer is approximately 3.6%. In still other embodiments, the compound remains bonded to the polymer for a period of at least 1, 2, 3, 4, 6, 8, 10, 15, 20, or 30 weeks in phosphate-buffered saline at pH 7.4 and 37° C. In yet other embodiments, the compound remains bonded to the polymer for a period of at least 1, 2, 3, 4, 6, 8, 10, 15, 20, or 30 weeks in vivo, such as in the blood of a subject.

The method can be used with any biocompatible urethane polymer; preferred urethanes are polycarbonate urethanes containing carboxylic acid functional groups or other functional groups that permit bonding between the groups and acid groups on the adsolved organic compound. The therapeutically active organic compound applied to the urethane polymer is preferably a small molecule (mw<1000), and can be an antifungal agent, an antiviral agent, an antiseptic agent, an antibiotic, or a combination thereof. The antibiotic used in this method can include quinolone. Inorganic therapeutically active compounds such as silver, silver salts, gold, or gold salts may also be bonded to the polymers of the present invention. This bonding may involve an ionic interaction between the compound and the polymer.

In various embodiments, the therapeutically active organic compound includes a carboxylic acid group. In other embodiments, the therapeutically active organic compound includes an aryl group, which may enhance the interaction between the compound and the polymer. Desirable aryl groups include monovalent aromatic hydrocarbon radicals consisting of one or more rings in which at least one ring is aromatic in nature, which may optionally be substituted with one of the following substituents: hydroxy, cyano, alkyl, alkoxy, thioalkyl, halo, haloalkyl, hydroxyalkyl, nitro, amino, alkylamino, diakylamino, or acyl. Other suitable aryl groups include heteroaryl groups in which one or more carbons in a ring have been replaced with another atom, such as nitrogen, sulfur, or oxygen. Yet other suitable aryl groups include a phenyl, benzyl, or benzoyl moiety that is either unsubstituted or that contains one or more nitro, halo (e.g., chloro, fluoro, iodo, or bromo), aryl, (e.g., phenyl or benzyl), alkyl, alkoxy (e.g., methoxy), or acyl (e.g., acetyl or benzoyl) substituents.

The polymer which has adsorbed an effective amount of the therapeutic compound can be used in any medical application in which biocompatible polymers are used, and in which infection or other complications are to be avoided. Examples are used as a wound dressing or implantable device. Preferred devices are catheters, vascular grafts, artificial hearts, other artificial organs and tissues, blood filters, pacemaker leads, heart valves, and prosthetic grafts. In various embodiments, the polymer is non-toxic, does not contain an exogenous binder agent, and/or does not induce clot formation. The polymers can also be used in commercial products that are desirably antibacterial, antiviral, or antifungal, e.g. shower curtains, clothing, and foam cushions.

### BRIEF DESCRIPTION OF THE DRAWINGS

FIG. 1 is an illustration of the molecular structure of commonly used dyes and antibiotics. Disperse Blue 1 and Orange Disperse Dye (A and B, column 1) have characteristics comparable to the fluoroquinolone antibiotics cipro and oflox (A and B, column 2). Similarly, C.I. Direct Blue 106 (C, column 1) has chemical features similar to the antibiotic tetracycline (C, column 2).

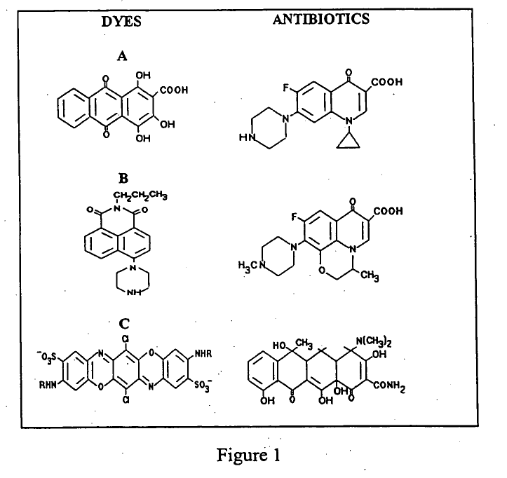

FIG. 2 is a schematic illustration of adsorption isotherms for Nernst, Langmuir, and Freudich distributions.

FIG. 3 is a schematic illustration of the dyeing apparatus.

FIG. 4 is a graph of absorbance versus concentration for cipro at 276 nm.

FIG. 5 is a graph of cipro concentration before and after dyeing versus dyeing pH.

FIG. 6 is a graph of cipro concentration before and after dyeing versus liquor ratio.

FIG. 7 is a graph of cipro concentration before and after dyeing versus applied cipro concentration.

FIG. 8 is a graph of the concentration of cipro on cPU (polyurethane-A) after dyeing versus applied cipro concentration.

FIG. 9 is a graph of cipro concentration versus dyeing temperature.

FIG. 10 is a graph of cipro concentration versus dyeing time.

FIG. 11 is a graph of the concentration of cipro released from dyed cPU, dyed bdPU, and dipped cipro versus time.

FIG. 12 is a picture of the zone of inhibition formed by cipro-dyed cPU segments that were embedded in agar plates streaked with a solution of S. epidermidis.

FIG. 13 is a graph of the zone of inhibition size versus time for dyed cPU, dyed bdPU, a standard cipro sensi-disc, and cipro dipped cPU.

FIG. 14 is a graph of the zone of inhibition size versus time for cipro-dyed cPU for which the wash buffer was either changed or not changed and for a standard cipro sensi-disc.

FIG. 15 is a graph of the concentration of cipro absorbed by the fiber [Cipro]_{f} versus the concentration of cipro in solution [Cipro]ₛ under the dyeing conditions listed in Table 7.

FIG. 16 is a graph of 1/[Cipro]_{f} versus 1/[Cipro]ₛ using the data from FIG. 15. This curve has an R-square value equal to 0.9229, suggesting that the absorption of cipro by cPU is based on a “site” mechanism and follows a Langmuir distribution.

FIG. 17 is a graph of log [Cipro]_{f} versus log [Cipro]ₛ using the data from FIG. 15.

FIG. 18 is a schematic illustration showing possible interactions between the carboxylic groups of cPU and those of cipro.

### DETAILED DESCRIPTION

This invention features a method of applying a therapeutically active organic compound to a urethane polymer, preferably are containing a functional group within the polymer backbone. This method involves incubating the polymer with the compound in solution under conditions that result in adsorption of the compound from solution by the polymer. We have shown that the fluoroquinolone antibiotic ciprofloxacin (cipro) was preferentially absorbed from an aqueous solution by a medically-useful polycarbonate-based polyurethanes containing a carboxylic functional group, i.e. that dyeing took place. Because of their good tissue and blood compatibility, polyurethanes are an important family of biomaterials. They are frequently used for implantable devices, including heart valves, artificial organs, blood filters, catheters, wound dressings, pacemaker leads, and prosthetic grafts. They are segmented polymers, formed from diisocyanates and polyols. Early biomedical polyurethanes were polyether-based polymers. Although they had excellent stability in vitro, they showed surface degradation in vivo resulting from several degradative reactions. The development of polyurethanes using polycarbonate-based diols overcame these problems and they are widely used today. A typical material is formed from poly(1,6-hexoyl-co-1,2-ethyl-carbonate)diol and 4,4′-diphenylmethane diisocyanate (MDI), with 1,4 butanediol as the chain extender. This polyurethane demonstrated not only improved compatibility with blood but also maintained the biodurability of the basic polycarbonate polyurethane.

Based on this biodurable formulation, a polycarbonate urethane with carboxylic acid sites (cPU) extending from the polymer backbone to match those functional groups present on the hydrolyzed polyester has been previously synthesized. Carboxylic acid groups were incorporated into the polymer by using the chain extender 2,2-bis(hydroxymethyl)propionic acid in place of 1,4 butanediol (bdPU) (Phaneuf et al., J. of Biomatrials Applications 12:100, 1997).

The diffusion of dyes into fibers requires “access” and depends on the swelling of hydrophilic fibers in the application medium (usually aqueous) or the segmental mobility of hydrophobic polymer chains at the application temperature. Medical polyurethanes typically have a low glass transition temperature (Tg), and comparison with the only polyurethane textile fiber, spandex, suggests that these materials would be readily accessible to a dye or an antibiotic.

Commercial antibiotics do not have ideal dyeing behavior, as compared to dyes, and that is beneficial in this invention. A relatively low affinity (representing poor fastness for a dye) results in a good leaching rate of antibiotic from the cPU, thus providing sustained antimicrobial activity. Such leaching is essential for antimicrobial activity: antibiotic durably incorporated within a polymer structure would be ineffective. Additionally, antibiotic uptake can be optimized so that the dyed cPU material possesses controlled sustained antibiotic release.

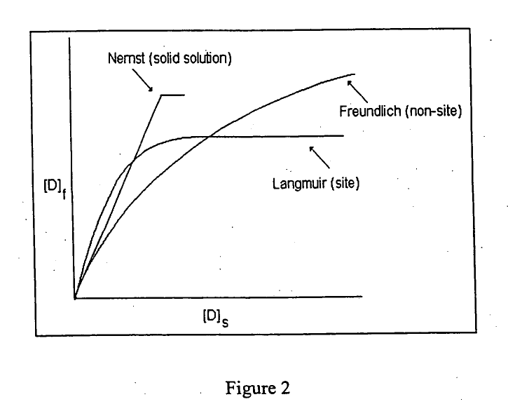

When cPU was tested with a range of dyes (Example 1), cPU could be dyed with both basic dyes and disperse dyes, suggesting that Langmuir and Nernst equilibria might be involved in the dyeing of cPU (FIG. 2). A range of dyeing conditions, including pH, temperature, concentration of cipro, liquor ratio, and dyeing time, was examined in order to obtain maximum uptake of cipro by cPU. The optimum conditions for the uptake of cipro were determined to be at a liquor ratio of 20:1, a pH of 8.6, and a temperature of 55° C. (Example 2). An equilibrium uptake was established at a time of 3.5 hours (Example 2). Dyeing conditions are required for this uptake of cipro because infection-resistance is lost within 4 hours when cPU is exposed to the antibiotic under dyeing conditions minus the heat (Example 3). In contrast, cipro-dyed cPU showed a sustained zone of inhibition up to 9 days, which correlated with the spectrophotometric data (Examples 3 and 4). This ready release of cipro, albeit over a long period of time, corresponds to the low standard affinity calculated below (Example 6). Stringent washing of the cipro-dyed surface resulted in greater release of the antibiotic (9 days) as compared to segments in which the wash bath was unchanged (>9 days) (Example 5). The described procedures for optimizing cipro dyeing can be used to optimize adsorption of any other organic molecule to urethane polymer.

The optimum conditions dyeing conditions from Example 2 were used to apply a range of cipro concentrations to cPU and derive the sorption isotherm, which suggested a Langmuir distribution (Example 6). The saturation value (0.45 g/kg) corresponded closely with the known concentration of carboxylic acid groups in cPU, indicating again that the carboxylic acid groups are the “sites” for dyeing. It is postulated that the mode of interaction between cipro and these carboxylic acid groups is hydrogen-bonding between acid groups. The lack of uptake by the corresponding polyurethane lacking carboxylic acid groups is further evidence for this. Using the value for the distribution coefficient, K, obtained in Example 6 and making a number of assumptions (for example, that the interaction is nonionic, and that activities are equal to concentrations) a value for the standard affinity of cipro for this cPU of 4.69 kJ/mol was obtained. This value is the same order of magnitude as, but lower than, the usual range of quoted values for standard affinities for a wide range of dye fiber systems, and corresponds to the comparatively low exhaustion obtained here. If this calculation could be suitably refined, the attraction between antibiotic and fiber could be correlated with the rate of release, allowing the degree and time of subsequent antimicrobial activity to be predicted.

These results demonstrate that cipro can be applied to ionic polyurethane via dyeing, which does not rely on exogenous binders or agents. The dyed urethane polymer possessed a slow, sustained release of the antibiotic. This binding can be optimized and the antibiotic/material interaction characterized using standard textile principles. This novel dyed urethane polymer can be applied as a coating to established implantable devices such as catheters, vascular grafts, artificial hearts, wound dressings, sutures, catheters, artificial heart, or heart valves. Additionally the dyed polymer can be employed as the main material to design a novel infection-resistant device. Other antibiotics, antiseptic, or antifungal agents or possible combination thereof may be applied using this technology since these agents should have structural stability under dyeing conditions (temperatures below 90° C.). Additionally, this work has use in commercial products such as shower curtains, clothing or foam cushions were bacteria and fungi presence is not desired.

This method of applying a therapeutically active organic compound to a urethane polymer containing a functional group within the polymer backbone holds several key advantages over the antibiotics bound in other studies: the antibiotic attaches to the polyurethane without molecular modification, thus retaining full antimicrobial activity; no cross linking agents are needed, avoiding concerns over drug carrier toxicity, biocompatibility, and mutagenicity; antibiotic leaching is controlled and sustained, a broader spectrum of bacteria are killed using quinolone antibiotics as compared to antiseptic agents; and quinolone antibiotics are less prone to creating infection-resistance as compared to other antibiotics due to broad spectrum antimicrobial activity.

### EXAMPLE 1

### Application of Various Dyes to Ionic Polycarbonate Based Urethane (cPU) Via Textile Dyeing Technology in Order to Characterize Dye Uptake by cPU Using Defined Interactions

An experiment was carried out to determine which, if any, classes of dye will dye cPU and bdPU. Based on these results, the type of binding forces between the dyes and polyurethane, and the dyeing properties of the polyurethanes were determined. The dyes selected for the study were: direct dyes: C.I. Direct Blue 25, C.I. Direct Blue 199; acid dyes: C.I. Acid Blue 127, C.I. Acid Blue 45, C.I. Acid Blue 83; basic dyes: C.I. Basic Blue 41, C.I. Basic Blue 45, C.I. Basic Blue 62; disperse dyes: C.I. Disperse Blue 165, C.I. Disperse Blue 172; and reactive dyes: C.I. Reactive Blue 29, C.I. Reactive Blue 225.

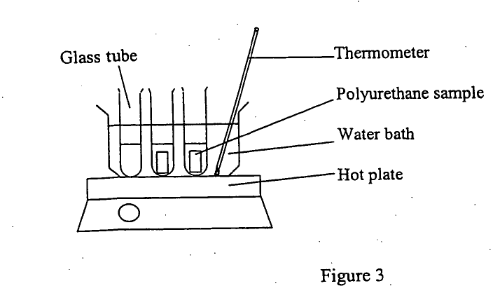

Dyeing was conducted with the individual polyurethane specimen in a glass tube with an antibiotic solution. The glass tube was then set in a water bath. A hot plate was used to achieve the desired temperature (FIG. 3). For all dyeing, the liquor ratio was 100:1, and the amount of dye was 10% of the weighed polymer/fiber (owf). The temperature was raised from 21° C. to 65° C. in 20 minutes and maintained at that temperature for 45 minutes (cPU deforms at higher temperatures than 65° C.). For direct dyes, dyebaths with and without 20 g/l salt were tested. For acid dyes, dyebath pH values of 6.0, 3.3 and 2.5 (pH achieved with either acetic acid or sulfuric acid) were used.

After dyeing, the polyurethane samples were rinsed in de-ionized water. The depths of color on polyurethane A and B samples (K/S) were evaluated by Datacolor CS-5 reflectance spectrophotometer and software. Since the polyurethanes were transparent, a white backing was used for each measurement.

The K/S (equivalent to color intensity) values of different classes of dyes on the dyed polyurethanes are shown in Table 1. The K/S values of both polyurethanes dyed with direct and reactive dyes are very low, ranging from 02-0.07. There was almost no color dyed on the two materials. The presence of salt had little effect. cPU dyed with C.I. Acid Blue 83 at pH 2.5 has a K/S of 4.2588, which could be due to the protonation of urethane groups under the low pH conditions. All other acid dyes produced little color (K/S values less than 1.0) on the cPU and bdPU polyurethanes.

The two disperse dyes produced higher K/S values than shown by the acid, direct and reactive dyes on the two polyurethanes (6.0 for bdPU and 1.0 for cPU). The higher K/S for bdPU is probably due to hydrophobic property of this material. There are affinities between disperse dyes and two polyurethanes. Basic dyes produced very little color on bdPU (K/S values less than 0.2). In contrast, the highest K/S values of the study (two over 10.0) were obtained between the three basic dyes and cPU. This is understandable on the basis of ionic interaction between the cationic dye and the anionic carboxyl groups of cPU.

The above results give an overall picture of the dyeing properties of polyurethanes A and B. The ability to dye cPU with basic dyes and with disperse dyes suggests that Langmuir and Nernst equilibria might be involved in the dyeing of cPU (FIG. 2).

### EXAMPLE 2

### Application and Optimization of the Quinolone Antibiotic Cipro to cPU Films Via Dyeing at Various Conditions Such as Temperature, pH, Liquor Ratio, Cipro Concentration and Reaction Time

For each dyeing condition, both cPU and bdPU polyurethanes were evaluated. An additional control consisting of a blank dyebath (prepared as in dyeing but with no cPU added) was evaluated. This control was performed in order to check if the dyeing conditions affected the stability of cipro.

In order to obtain maximum exhaustion of cipro on polyurethane, different dyeing conditions were tested. The basic experiment used a liquor ratio of 20:1, a cipro concentration of 2% owf, a pH of 8.6, a dyeing temperature of 45° C., and a dyeing time of 3.5 hours. pH, temperature, cipro concentration, liquor ratio and dyeing time were varied individually. The polyurethane samples were removed after dyeing. The pH of the remaining dyebaths were measured and re-adjusted to 3.73 using 10% acetic acid. A dilution factor of 125 fold was used (0.2 ml cipro solution was dissolved in water and brought up to a total volume of 25 ml) for measuring the absorbance of the dyebath at 276 nm and 25° C. A range of pH values (3.73, 4.84, 5.63, 6.61, 7.63, and 8.64) was assessed. The pH of the unadjusted dyebath at liquor ratio 20:1 and 2% owf cipro concentration was 3.73. To achieve pH's of 4.84, 5.63, 6.61, 7.63, and 8.64, NH₄OH (10%) and NH₄Cl (10%) buffer solution (pH=9.92) was used. A series of liquor ratios from 10:1, 20:1, 40:1, 60:1, 80:1, to 100:1 were conducted to determine the ratio for maximum exhaustion of cipro. Cipro concentrations of 0.5% owf, 1.0% owf, 2.0% owf, and 4.0% owf were evaluated. Dyeing temperatures of 25° C., 35 C, 45° C., 55 ° C., and 65° C. dyeing times of 15 minutes, 30 minutes, 45 minutes, 1 hour, 1.5 hours, 2 hours, 3.5 hours and 4.5 hours were examined.

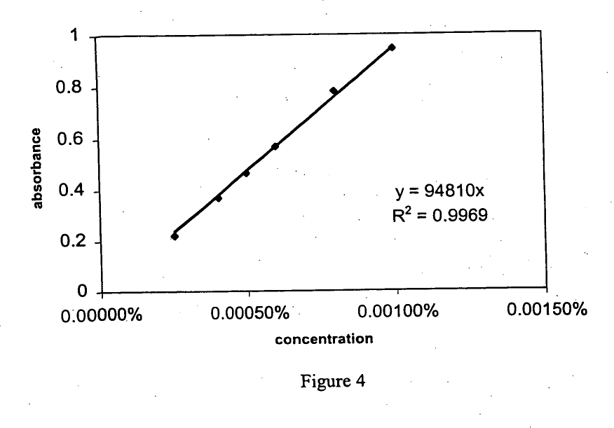

To obtain the relationship between the concentration and the absorbance of cipro, a solution of cipro was made and diluted to get a series of final concentrations of 0.00025%, 0.0004%, 0.0005%, 0.0006%, 0.0008%, and 0.001%. The pH values of this series of concentrations of cipro were all adjusted to 3.73 using 10% acetic acid to prevent sedimentation of cipro at some pH values which would effect the absorbance readings. The maximum absorbance wavelength was determined to be 276 nm. The absorbance of each concentration was read at this wavelength and an extinction coefficient for the ciprofloxacin then was calculated using the Beer-Lamber equation,
A_{λmax}=k c(g/l)
The value of the absorbance coefficient k at a given wavelength (λmax) was determined by measuring the slope of a plot of a series of absorbance (A) measurements of solutions of known concentration (c). Having determined the value of k, any concentration of ciprofloxacin in this study can be determined. FIG. 4 shows the relationship of absorbance and concentration for cipro at pH 3.73, 25° C., and λmax 276 nm. The slope of the plot is 94810, which is the absorbance coefficient of the cipro. An R-square value of 0.9969 indicates a good linear relationship between absorbance and concentration

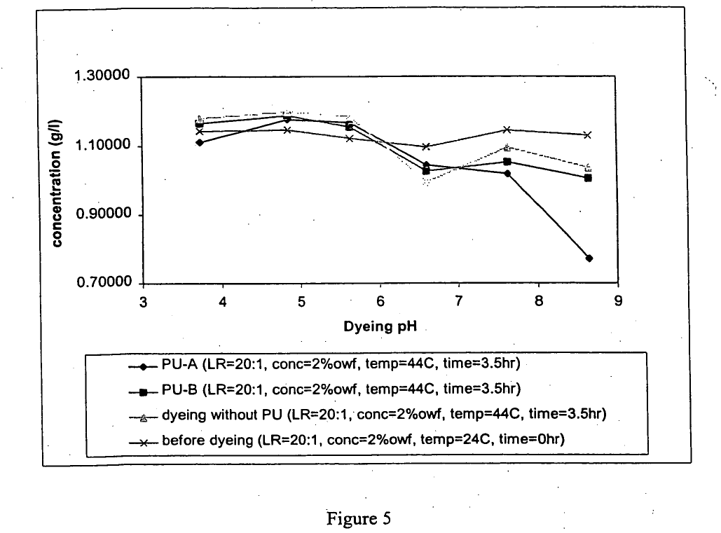

The cipro concentration before and after dyeing with cPU and bdPU at different pH is shown in FIG. 5. In previous studies, cipro was shown to be stable to the dyeing conditions employed. There was little difference between the concentrations before dyeing and after the dyeing with no polyurethane present. The presence in the bath of bdPU did not cause a drop in cipro concentration at any pH, indicating that bdPU was not absorbing the antibiotic. cPU similarly did not absorb cipro from pH 3.7 to 7.6. However, at pH 8.6 the concentration of cipro in the bath dropped dramatically during dyeing. The change in pH can only be caused by absorption of the cipro by cPU. Approximately 32% of the cipro present was absorbed by cPU. The difference between the two polyurethanes under the same dyeing conditions is due to the presence of —COOH groups in cPU. Based on this result, a pH of 8.6 was chosen to achieve a maximum exhaustion of cipro. The pH values of the baths before and after dyeing are shown in Table 2.

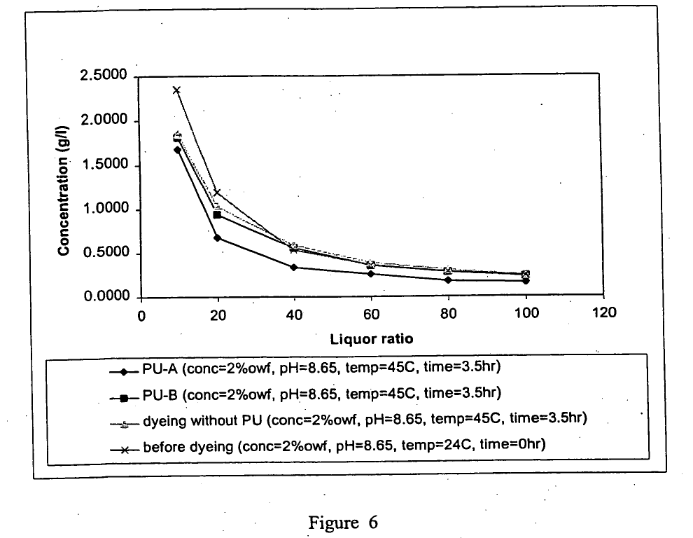

At all liquor ratios examined, from 10:1 to 100:1, the concentration of cipro in baths with cPU were lower than those of bdPU and those without polyurethane, representing the uptake of cipro by cPU (FIG. 6). The greatest relative uptake occurred at a liquor ratio 20:1. Again there was no difference between the cipro concentration of baths with bdPU and without polyurethane, therefore no cipro was absorbed by bdPU. The overall pH values of the bath after dying for cPU were lower than that of bdPU and without polyurethane (Table 3).

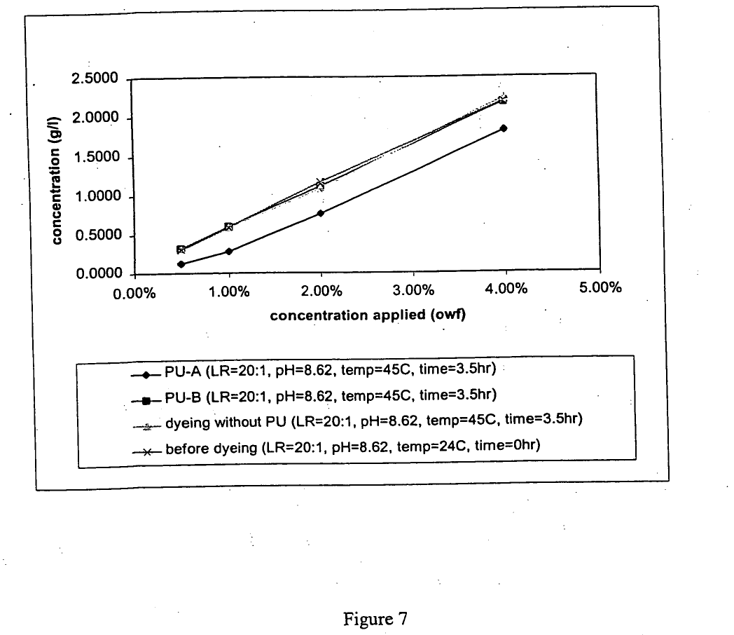

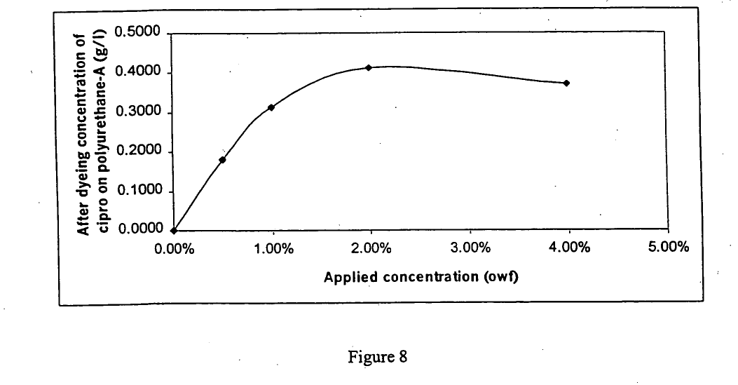

The concentrations of cipro in the bath at the end of dyeings with cPU were lower at all applied concentrations (0.5% to 4%) than for baths with bdPU and baths without polyurethane (FIG. 7). The exhaustion of cipro is highest (about 60%) at 0.5% owf. The exhaustion is defined as the ratio of amount of dye on fiber at the end of dyeing to the amount of dye applied at the start of dyeing. A decrease in exhaustion with increase in applied concentration is unvaryingly observed in dyeings. There was no concentration change in the baths with no polyurethane or those with bdPU indicating again that bdPU does not absorb cipro. As can be seen in FIG. 8, an applied cipro concentration of 2.0% owf resulted in the maximum amount of cipro uptake by cPU. Greater applied cipro concentrations did not increase cipro uptake. The pH values of the baths before and after dyeing (Table 4) show that the pH of the bath after dying for cPU is lower than that bdPU and without polyurethane.

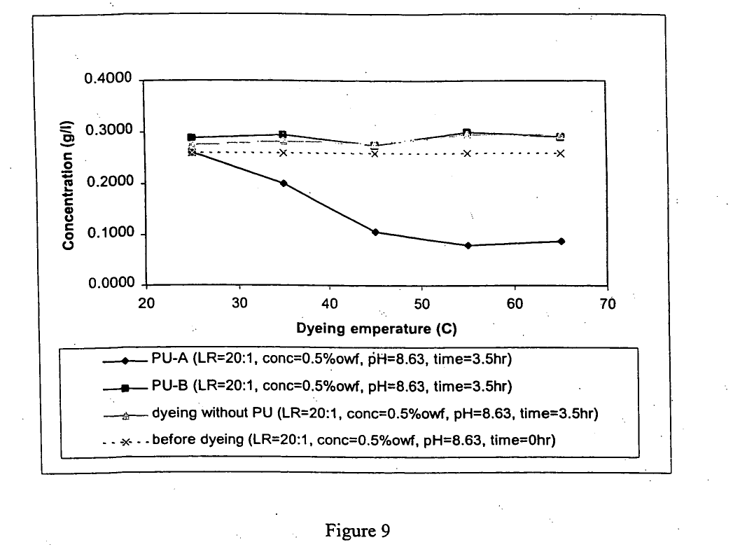

The concentration of cipro in dyebaths after dyeing with cPU was less than in baths bdPU and without cPU temperatures over 35° C. (FIG. 9). The largest difference occurs at a dyeing temperature of 55° C. (70% of the ciprofloxacin is taken up by cPU) which was thus chosen as the optimum. No cipro was absorbed by bdPU. Table 5 shows the pH values of the baths before and after dyeing for different dyeing temperatures.

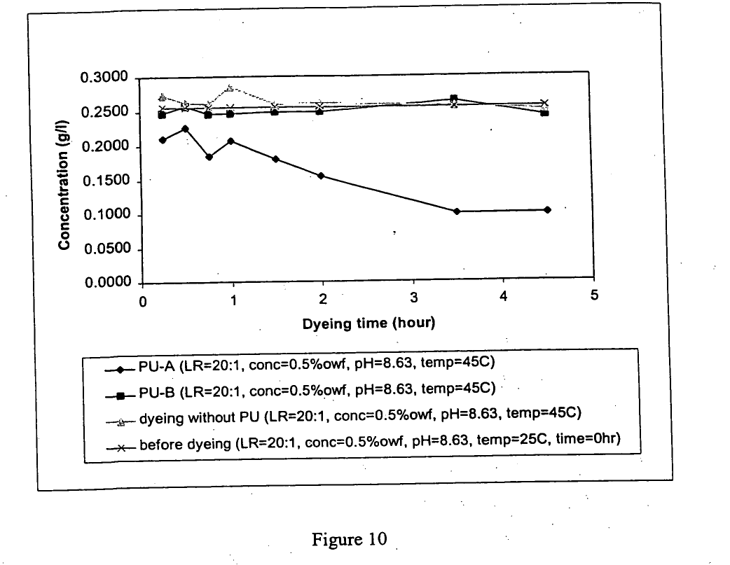

When the dyeing time was varied, cipro concentrations in baths with cPU were lower than that dyed with bdPU and those without polyurethane (FIG. 10). The lowest absorbance was obtained at a dyeing time of 3.5 hours (about 61% exhausted) and this time was selected for optimum exhaustion. No cipro was taken up by bdPU. The pH values of the baths before and after dyeing for different dyeing times are listed in Table 6.

### EXAMPLE 3

### Determination of Cipro Release from cPU Film Segments as Determined by Spectrophotometric Analysis

Segments (1 cm²) of bdPU, cipro-dipped, and dyed cPU were cut from 9 cm (diameter) circular pieces (3 segments tested per time interval per test condition). Time intervals of ranging from 1 hour to 11 days were evaluated for each treatment. Segments that were unwashed served as the T=0 control. All segments were placed into sterile 15 ml Falcon tubes. Phosphate-buffered saline (0.1 M monobasic sodium phosphate, 0.05 M sodium chloride, pH 7.4; PBS) was prepared and sterilized via filtration. PBS (5 ml) was then added to each time interval and tubes were placed onto an inversion mixer that rotated at 33 rpm at 37° C. At each time interval, PBS was removed from the samples and a fresh 5 ml PBS was added. A cipro standard curve was derived using cipro at pH 7.4, with antibiotic concentrations ranging from 0.010 to 80 μg/ml. Cipro absorbance versus concentration was plotted at 332 nm (linear coefficient=0.998). Using this standard curve, cipro release μg/ml) from the 3 segments at each time intervals was determined.

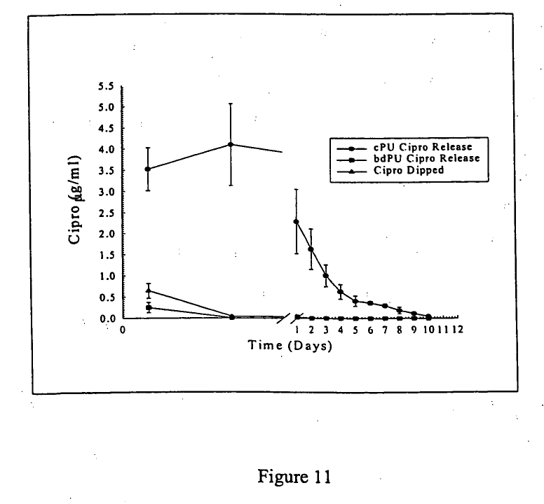

Cipro release from both cipro-dipped and bdPU segments dyed with the antibiotic occurred within 4 hours of washing (FIG. 11). The cipro concentration released from these segments at the 1 hour wash was significantly lower than the cipro-dyed cPU, indicating that antibiotic uptake by each control was lower than the dyed segments. Cipro uptake by cPU dipped into the antibiotic was greater than bdPU exposed to the antibiotic under dyeing conditions, demonstrating some low-level surface interaction at room temperature. The cipro-dyed cPU segments had significant levels of antibiotic released over 9 days followed by minimal release at 10 days.

### EXAMPLE 4

### Assessment of Antimicrobial Activity of Cipro-Dyed cPU Segments Against S. epidermidis Using a Zone of Inhibition Study

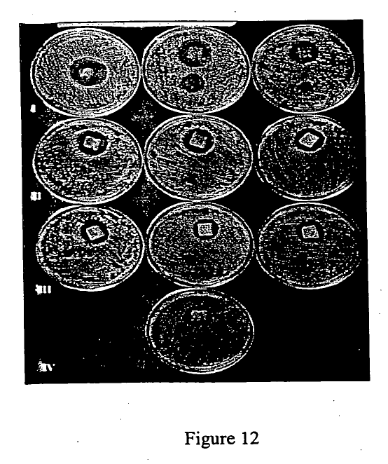

An inoculum of S. epidermidis (ATCC # 33501) was thawed at 37° C. for 1 hour and 1 μl of this stock was added to 10 ml of Trypticase Soy Broth (TSB). This S. epidermidis solution was incubated overnight at 37° C. and had an approximate bacterial concentration of 10⁸ colony forming units (cfu)/ml. From this solution, 10 μl was streaked onto agar plates (BBL Trypticase Soy Agar+0.5% dextrose) to create a bacterial lawn. Segments from the spectrophotometric study were then embedded into the agar (n=3 segments tested per time interval per treatment) and placed into a 37° C. incubator overnight. Standard 5 (g cipro Sensi-Discs (n=2) were also embedded at each time interval. The zone of inhibition of each piece was determined, taking the average of 3 individual diameter measurements (FIG. 12). Zone size (mm) over time was then determined. These zones were compared to the spectrophotometric data to determine any correlations between these two methods. Samples with no zone of inhibition were transferred to sterile 50 ml polypropylene tubes containing 30 ml of TSB. Sonication of samples was achieved at 60 Hz for 5 minutes in an ice bath (Tollefson et al., Arch. Surg. 122:38, 1987). Sonicated solutions (100 μl) were backplated onto an agar plate and examined after 24 hours to determine the presence of adherent bacteria on the segments.

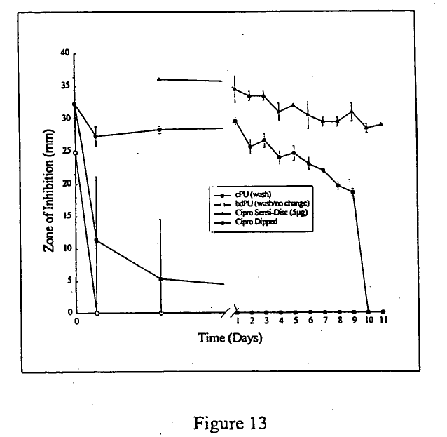

The standard cipro discs had consistent release, demonstrating the reliability of the technique. Cipro-dyed bdPU had no zone of inhibition after 1 hour of washing. Cipro-dipped cPU had antimicrobial activity that remained for less than 24 hours. In contrast, the cipro-dyed cPU possessed antimicrobial activity for 9 days, with no detectable activity at 10 days (FIG. 13). Backplates of all samples with no zone resulted in bacterial growth. These results correlated with the spectrophotometric studies that indicated cipro-dyed segments had significant antibiotic release compared to the controls, with minimal release at 10 days (0.05 μg/ml for 3 segments). Controls also had no zone of inhibition below this threshold. Thus, three segments releasing less than 0.10 μg/ml will not possess antimicrobial activity as indicated by zone of inhibition.

### EXAMPLE 5

### Evaluation of Cipro-Dyed cPU Under Changed and Unchanged Wash Conditions

S. epidermidis streaked agar plates were used to determine the effects of varying volume exposure to the segments. Time intervals of ranging from 1 hour to 11 days were evaluated for each treatment. Segments that were unwashed served as the T=0 control. All segments were placed into sterile 15 ml Falcon tubes. Sterile PBS (5 ml) was then added to each time interval and tubes were placed onto an inversion mixer that rotated at 33 rpm at 37° C. In one set of tubes, PBS was removed from the samples at each time interval and a fresh 5 ml PBS was added. For the other set, PBS was not changed and was removed prior to embedding the segments. Standard 5 μg cipro Sensi-Discs (n=2) were embedded at each time interval. The zone of inhibition of each piece was determined, taking the average of 3 individual diameter measurements. Zone size (mm) over time was then determined. Samples with no zone of inhibition were transferred to sterile 50 ml polypropylene tubes containing 30 ml of TSB. Sonication of samples was achieved at 60 Hz for 5 minutes in an ice bath. Sonicate solutions (100 μl) were backplated onto an agar plate and examined after 24 hours to determine the presence of adherent bacteria on the segments.

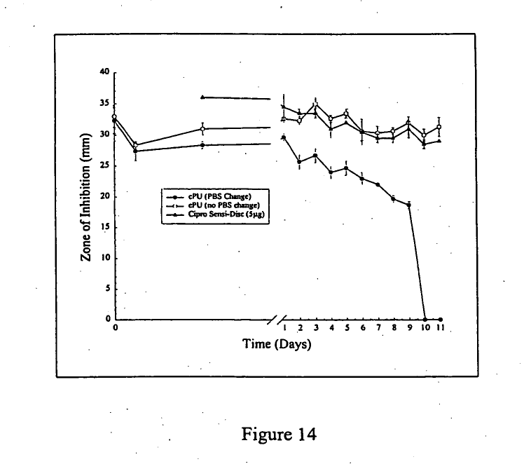

Cipro-dyed cPU in which the wash buffer was changed possessed antimicrobial activity for 9 days, with no detectable activity at 10 days (FIG. 14). In contrast, Cipro-dyed cPU in which no PBS change occurred had zone sizes that remained consistent over the 11 days evaluated. Backplates of all samples with no zone resulted in bacterial growth. This study suggests that cipro release from the blood-contacting surface of a medical device will be greater because it is washed with blood than the portion of the device contained within the epidermidis and subcutaneous areas.

### EXAMPLE 6

### Dyeing Thermodyamic Study

Once optimum conditions for the application of cipro were established, they were used to apply a range of cipro concentrations to cPU and thereby derive a dyeing isotherm (Table 7). The absorbance of the cipro solution was measured under the same conditions each time: the pH was adjusted to 3.75, the dilution factor was 0.2/25, and the temperature was 25° C. The initial and final concentrations of cipro in solution and cipro in cPU were determined using the absorbance coefficient measured in Example 2. The amount of cipro in cPU was computed using the initial concentration minus the final concentration of cipro in the dyebath.

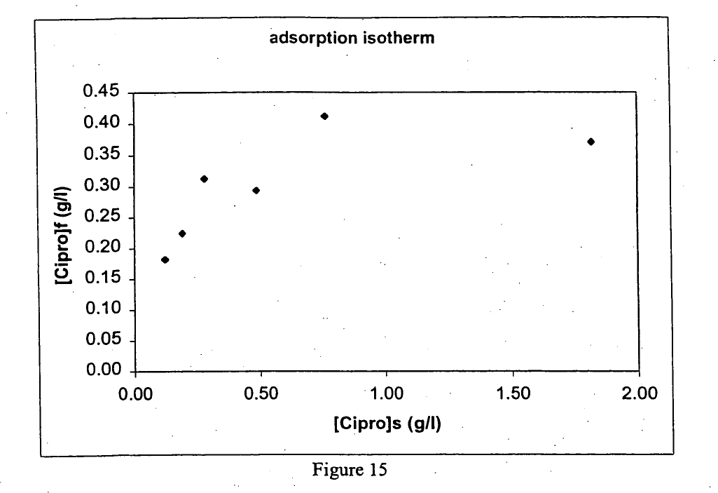

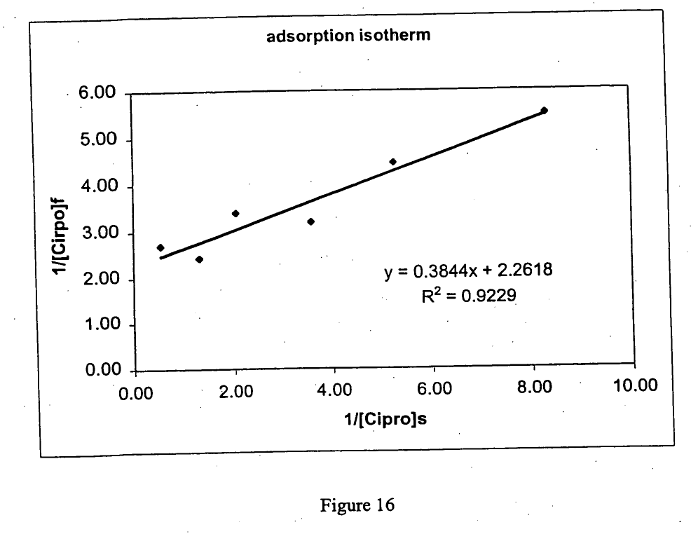

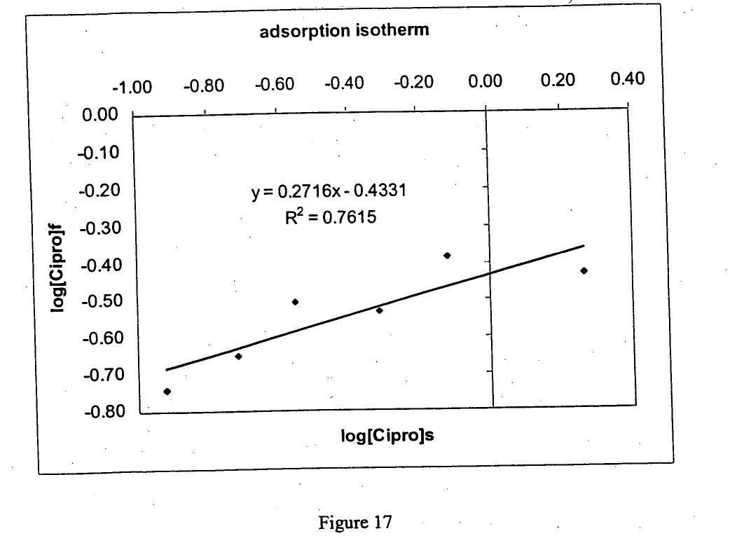

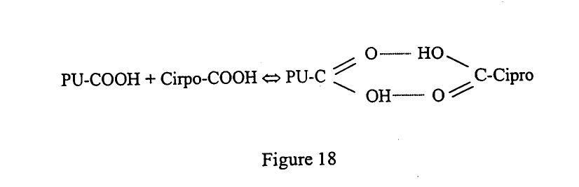

The adsorption isotherm plots are shown in FIGS. 15-17. The curve in FIG. 15 is very much like a Langmuir distribution. In FIG. 16, the plot of 1/[cipro]_{f} versus 1/[cipro]ₛ, with an R-square value equal to 0.9229, again suggests that the absorption of cipro by polyurethane-A is based on a “site” mechanism and follows a Langmuir distribution. Since the only difference between cPU and bdPU is the presence of carboxylic acid groups in A, it is postulated that these groups form the “sites” to which the cipro is attached. It is possible that the carboxylic acid groups in cPU are associated with the carboxylic acid groups in cipro through hydrogen-bonds (FIG. 18).

Based on the equation for a Langmuir distribution:
1/[cipro]_{f}=1/(K[S]_{f}[cipro]ₛ)+1/[S]_{f}
and the equation obtained from FIG. 16:
y=0.3844x+2.2618 (where y=1/[cipro]_{f} and x=1/[cipro]ₛ)
the saturation value of cipro on the cPU [S]_{f} is 0.4421 site/kg, and Langmuir isotherm distribution coefficient K is 5.8963.

At equilibrium, the standard affinity is
−Δμ°=RT ln (aₛ/a_{f})=RT ln K

where R is the gas constant (8.3143 J/K mol) and T is the absolute temperature (318.15 K) and K is the Langmuir isotherm distribution coefficient (5.8963). From above calculation, the value of standard affinity is 4.69 kJ/mol.

TABLE 1
The depth of shades (K/S) of cPU and bdPU with different classes of dyes

| | | Dyeing | K/S* | |
|---|---|---|---|---|
| Class of Dyes | Name of Dyes | Condition | cPU | BdPU |
| Direct Dyes | C.I. Direct Blue 25 | With salt | 0.0360 | 0.0653 |
| | | Without salt | 0.0286 | 0.0540 |
| | C.I. Direct Blue 199 | With salt | 0.0313 | 0.0336 |
| | | Without salt | 0.0389 | 0.0312 |
| Acid Dyes | C.I. Acid Blue 61 | pH = 2.5 | 0.6457 | 0.5607 |
| | | pH = 3.3 | 0.2492 | 0.2108 |
| | | pH = 6.0 | 0.2057 | 0.1855 |
| | C.I. Acid Blue 83 | pH = 2.5 | 4.2588 | 0.8942 |
| | | pH = 3.3 | 0.8807 | 0.4064 |
| | | pH = 6.0 | 0.9509 | 0.5580 |
| | C.I. Acid Blue 127 | pH = 2.5 | 0.4025 | 0.1413 |
| | | pH = 3.3 | 0.7395 | 0.1336 |
| | | pH = 6.0 | 0.1322 | 0.0759 |
| Basic Dyes | C.I. Basic Blue 41 | | 11.805 | 0.1647 |
| | C.I. Basic Blue 45 | | 6.7500 | 0.1647 |
| | C.I. Basic Blue 62 | | 10.821 | 0.1906 |
| Disperse Dyes | C.I. Disperse Blue 165 | | 5.1600 | 6.8605 |
| | C.I. Disperse Blue 172 | | 1.1467 | 1.2122 |
| Reactive Dyes | C.I. Reactive Blue 109 | | 0.0353 | 0.0302 |
| | C.I. Reactive Blue 160 | | 0.0285 | 0.0246 |
| | C.I. Reactive Blue 163 | | 0.0345 | 0.0322 |

*Measuring condition: ultra small area view, D65, CIELAB, 10 degrees

TABLE 2
pH of the baths before and after dyeing

| | Dyebath pH after dyeing | | |
|---|---|---|---|
| pH before dyeing | cPU | bdPU | no polyurethane |
| 3.73 | 3.77 | 3.74 | 3.74 |
| 4.84 | 4.85 | 4.87 | 4.87 |
| 5.63 | 5.52 | 5.52 | 5.46 |
| 6.61 | 6.16 | 6.10 | 6.19 |
| 7.63 | 6.94 | 6.97 | 7.18 |
| 8.64 | 7.46 | 8.00 | 8.02 |

TABLE 3
pH of the baths before and after dyeing
for dyeings at different liquor ratios

| Dyeing liquor | Dyebath pH | Dyebath pH after dyeing | | |
|---|---|---|---|---|
| ratios | before dyeing | cPU | bdPU | no polyurethane |
| 10:1 | 8.65 | 7.39 | 8.31 | 8.31 |
| 20:1 | 8.65 | 7.62 | 8.36 | 8.34 |
| 40:1 | 8.65 | 7.83 | 8.29 | 8.28 |
| 60:1 | 8.65 | 7.93 | 8.30 | 8.30 |
| 80:1 | 8.65 | 7.97 | 8.33 | 8.32 |
| 100:1 | 8.65 | 7.99 | 8.27 | 8.26 |

TABLE 4
pH of the baths before and after dyeing for dyeings at
different applied ciprofloxacin concentrations

| Applied dyeing | Dyebath pH | Dyebath pH after dyeing | | |
|---|---|---|---|---|
| concentrations | before dyeing | cPU | bdPU | no polyurethane |
| 0.5% owf | 8.62 | 7.67 | 8.21 | 8.23 |
| 1.0% owf | 8.62 | 7.66 | 8.25 | 8.26 |
| 2.0% owf | 8.62 | 7.55 | 8.26 | 8.29 |
| 4.0% owf | 8.62 | 7.46 | 8.24 | 8.24 |

TABLE 5
pH of the baths before and after dyeing
for dyeings at different temperatures

| Dyeing | Dyebath pH | Dyebath pH after dyeing | | |
|---|---|---|---|---|
| temperatures | before dyeing | cPU | bdPU | no polyurethane |
| 25° C. | 8.63 | 7.85 | 8.37 | 8.35 |
| 35° C. | 8.63 | 7.73 | 8.27 | 8.31 |
| 45° C. | 8.63 | 7.75 | 8.26 | 8.26 |
| 55° C. | 8.63 | 7.70 | 8.12 | 8.28 |
| 65° C. | 8.63 | 7.62 | 7.80 | 7.82 |

TABLE 6
pH of the baths before and after dyeing for different dyeing times

| | Dyebath pH | Dyebath pH after dyeing | | |
|---|---|---|---|---|
| Dyeing times | before dyeing | cPU | bdPU | no polyurethane |
| 0.25 hr | 8.63 | 8.15 | 8.38 | 8.39 |
| 0.50 hr | 8.63 | 8.14 | 8.36 | 8.35 |
| 0.75 hr | 8.63 | 8.06 | 8.35 | 8.36 |
| 1.00 hr | 8.63 | 8.07 | 8.33 | 8.31 |
| 1.50 hr | 8.63 | 8.05 | 8.30 | 8.31 |
| 2.00 hr | 8.63 | 8.01 | 8.30 | 8.31 |
| 3.50 hr | 8.63 | 7.86 | 8.24 | 8.26 |
| 4.50 hr | 8.63 | 7.89 | 8.25 | 8.24 |

TABLE 7
The dyeing conditions for obtaining an adsorption isotherm

| PH: | 8.62 | |
|---|---|---|
| Temperature: | 45° C. | |
| Time: | 4 hours | |
| Concentrations: | 0.25 | g/l |
| | 0.375 | g/l |
| | 0.50 | g/l |
| | 0.75 | g/l |
| | 1.0 | g/l |
| | 2.0 | g/l |

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within the claims.

1. A composition comprising a urethane polymer reversibly bonded to a therapeutically active compound via one or more reactive groups of said polymer.

2. The composition of claim 1, wherein said compound is bonded to said polymer via one or more carboxylic acid, amino, sulfo, and/or hydroxyl groups.

3. The composition of claim 2, wherein at least 25% of the carboxylic acid, amino, sulfo, and/or hydroxyl groups of said polymer are bonded to said compound.

4. The composition of claim 1, wherein said compound is directly bonded to said polymer.

5. The composition of claim 1, wherein said compound is bonded to said polymer through a hydrogen-bond.

6. The composition of claim 1, wherein said compound is bonded to said polymer for at least one day in phosphate-buffered saline at pH 7.4 and at 37° C.

7. The composition of claim 1, wherein said compound is bonded to said polymer for less than ten days in phosphate-buffered saline at pH 7.4 and at 37° C.

8. The composition of claim 1, wherein at said compound is bonded to said polymer or a period between one and ten days, inclusive, in phosphate-buffered saline at pH 7.4 and at 37° C.

9. The composition of claim 1, wherein said compound comprises a carboxylic acid group or an aryl group.

10. The composition of claim 1, wherein said compound is an antibiotic or an antifungal, antiviral, or antiseptic agent.

11. The composition of claim 10, wherein said antibiotic is a quinolone.

12. The composition of claim 11, wherein said quinolone is selected from the group consisting of ciprofloxacin, ofloxacin, norfloxacin, sparfloxacin, tomafloxacin, enofloxacin, lomefloxacin, pefloxacin, fleroxacin, and DU6859a.

13. A biocompatible device comprising a urethane polymer reversibly bonded to a therapeutically active compound via one or more reactive groups of said polymer.

14. The device of claim 13, wherein said device is selected from the group consisting of a catheters, vascular grafts, artificial hearts, blood filters, pacemaker leads, heart valves, and prosthetic grafts.

15. A wound dressing comprising a urethane polymer reversibly bonded to a therapeutically active compound via one or more reactive groups of said polymer.
